(19) United States
(12) Patent Application Publication
(10) Pub. No.: US 2007/0131573 A1
(43) Pub. Date: Jun. 14, 2007

(54) Container

(76) Inventor: Kathleen A. Boyles, New Market, VA (US)

Correspondence Address: MICHAEL N. HAYNES, 1341 HUNTERSFIELD CLOSE, KESWICK, VA, 22947, US

(21) Appl. No.: 10/938,466

(22) Filed: Sep. 10, 2004

(51) Int. Cl. A61B 19/02

(52) U.S. Cl. 206/438

(57) ABSTRACT

Certain embodiments can comprise an apparatus, comprising a non-garment container for a device. The container can comprise: a first portion; a second portion couplable to the first portion; and a releasable closure attached to the first portion and the second portion for closing a releasably closable first opening adjacent the first portion and said second portion. The container can define a second opening opposing the first opening. The first opening can close to form a plurality of sub-openings.

### BRIEF DESCRIPTION OF THE DRAWINGS

A wide variety of potential embodiments will be more readily understood through the following detailed description of certain exemplary embodiments, with reference to the accompanying drawings in which:

FIG. 1 is a front view of an exemplary embodiment of a container 1000;

FIG. 2 is a front view of an exemplary embodiment of a container 1000;

FIG. 3 is a rear view of an exemplary embodiment of a container 1000;

FIG. 4 is a side view of an exemplary embodiment of a container 1000;

FIG. 5 is a side view of an exemplary embodiment of a container 1000;

FIG. 6 is a perspective view of an exemplary embodiment of a container 1000;

FIG. 7 is a perspective view of an exemplary embodiment of a container 1000;

FIG. 8 is a perspective view of an exemplary embodiment of a container 1000;

FIG. 9 is a perspective view of an exemplary embodiment of a container 1000;

FIG. 10 is a perspective view of an exemplary embodiment of a container 1000;

FIG. 11 is a perspective view of an exemplary embodiment of a container 11000;

FIG. 12 is a perspective view of an exemplary embodiment of a system 12000; and

FIG. 13 is a flowchart of an exemplary method 13000.

### DEFINITIONS

When the following terms are used herein, the accompanying definitions apply.

adapted—made suitable for.

adjacent—adjoining with little or no intervening space.

adjoining—next to.

area—the measure of the space within a 2-dimensional region.

attached—joined or secured together.

auxiliary—supplemental.

auxiliary storage pocket—a pocket designed primarily for storage.

auxiliary storage volume—the interior volume of an auxiliary storage pocket.

bottom—in a normal operative embodiment, the lowest point of an object.

can—is capable of, in at least some embodiments.

circuit—a path or route the complete traversal of which without local change of direction or crossing the path requires returning to the starting point.

closable—capable of being closed.

close—alter an opening such that the opening becomes substantially obstructed.

closed—the result of closing.

closing—altering and/or manipulating a closure and/or opening such that the opening becomes substantially obstructed.

closure—a clasp, catch, and/or other closing device, such as a button, a snap, a zipper, a pin, a hook-and-loop fastener, and/or lacing, etc., and/or a portion thereof.

comprised—included in; a part of.

comprises—includes, but is not limited to.

comprising—including but not limited to.

container—something that at least partially, holds, carries, and/or encloses one or more items for transport, storage, and/or protection, etc.

couplable—capable of being coupled.

couple—to join, connect, and/or link two things together.

device opening—an opening through which a device and/or one or more items can be passed.

device opening area—the measure of a 2-dimensional region defined as a device opening.

dimensioned—constructed to a specific extent, scope or magnitude; such as a specific height, width, diameter, volume, area, etc.

enclose—surround on all sides.

enclosed—surrounded on all sides.

exit—a path leading through an opening and away from an interior of a container.

exterior—substantially non-interior.

fastened—attached firmly, but not fixedly.

fixedly—not releasably.

flexible device—an elongated non-rigid object, such as a cable, wire, antenna, tube, and/or hose, etc.

flexible medical tube—an elongated, non-rigid, fluid-conveyable object useable for a medical purpose.

flexible medical tube opening—an opening dimensioned such that a flexible medical tube may pass therethrough.

garment—an article of clothing. Would not include accessories such as jewelry, handbags, purses, shoulder bags, satchels, totes, backpacks, etc.

information device—any device capable of processing information, such as any general purpose and/or special purpose computer, such as a personal computer, workstation, server, minicomputer, mainframe, supercomputer, computer terminal, laptop, wearable computer, and/or Personal Digital Assistant (PDA), mobile terminal, Bluetooth device, communicator, “smart” phone (such as a Handspring Treo-like device), messaging service (e.g., Blackberry) receiver, pager, facsimile, cellular telephone, a traditional telephone, telephonic device, a programmed microprocessor or microcontroller and/or peripheral integrated circuit elements, an ASIC or other integrated circuit, a hardware electronic logic circuit such as a discrete element circuit, and/or a programmable logic device such as a PLD, PLA, FPGA, or PAL, or the like, etc. In general any device on which resides a finite state machine capable of implementing at least a portion of a method, structure, and/or or graphical user interface described herein may be used as an information device. An information device can include well-known components such as one or more network interfaces, one or more processors, one or more memories containing instructions (such as firmware and/or software), and/or one or more input/output (I/O) devices, one or more user interfaces, etc.

interior—of, relating to, or located on the inside.

item—anything that can be at least partially, protected, housed, transported, concealed, supported, carried, and/or enclosed, etc., by a container.

medical device—an object usable for monitoring, maintaining, and/or enhancing the health of a person.

medical device storage pocket—a pocket designed specifically for, but not limited to, at least partially, holding, protecting, concealing, storing, transporting, carrying, and/or supporting, etc., a medical device.

medical device storage volume—the interior volume of a medical device storage pocket, or the volume of the interior of a medical device storage pocket.

medical-device-hiding—substantially obstructing visibility of a medical device.

medical-device-supporting—substantially bearing the weight of a medical device as well as holding it in position to keep it from falling.

medivice—a medical device.

normal operative embodiment—when the apparatus is operated and/or used in a typical manner. For example, when a container is being carried by hand, worn on a shoulder, hung on a hook, and/or hung on an IV-stand, etc.

obstructed—a condition where passage through is stopped and/or impeded.

obstructing—significantly shutting out.

offset—in a location near to but distinguishable from a given point or area.

open—not substantially obstructed.

opened—in an open condition.

opening—(n) an unobstructed area allowing access to and/or visibility of the interior of a container.

opening—(v) altering and/or manipulating a closure and/or opening such that the opening becomes substantially unobstructed.

opposing—aligned or positioned in an opposite direction from.

panel—a part of an item. Can be visually and/or physically distinguishable and/or non-distinguishable.

partially—to a certain extent but not totally.

pass—to move through and beyond without local change of direction.

perimeter—a circuit.

plurality—the state of being plural and/or more than one.

pocket—a container that at least partially encloses its contents.

portable—capable of being carried and/or moved with ease.

portion—a part of an item. Can be visually and/or physically distinguishable and/or non-distinguishable.

releasable—capable of being freed from a binding, fastening, and/or other restraint.

releasably closable—capable of being repeatedly and non-destructively closed and opened.

soft—not rough, abrasive, or harsh to the touch.

storage—a space for reserving and/or putting away goods, items, devices, objects, etc.

storage bag—a type of storage container.

storage container—a container that provides storage.

storing—reserving and/or putting away goods, items, devices, and/or objects, etc., in a container, storage, pocket, and/or bag, etc.

strap—a strip of material that can be constructed for grasping with a hand, placing over a shoulder, hanging on an IV pole, and/or hanging on a hook, etc.

sub-opening—a smaller opening created when a larger opening is closed.

substantially—to a majority extent. More so than not. For the most part.

support—to bear at least a substantial portion of the weight of.

transport—to move from one place to another.

tube opening area—the measure of the 2-dimensional region defined as a tube opening.

unabrasive—defining a low abrasiveness index.

underside—in a normal operative embodiment, a ground-facing side.

unfastened—not fastened.

volume—the capacity of the amount of space occupied by a three dimensional object or region of space measured in cubic units.

### DETAILED DESCRIPTION

Certain exemplary embodiments comprise a system comprising: a non-garment container for storing and/or transporting a medical device and/or one or more other items.

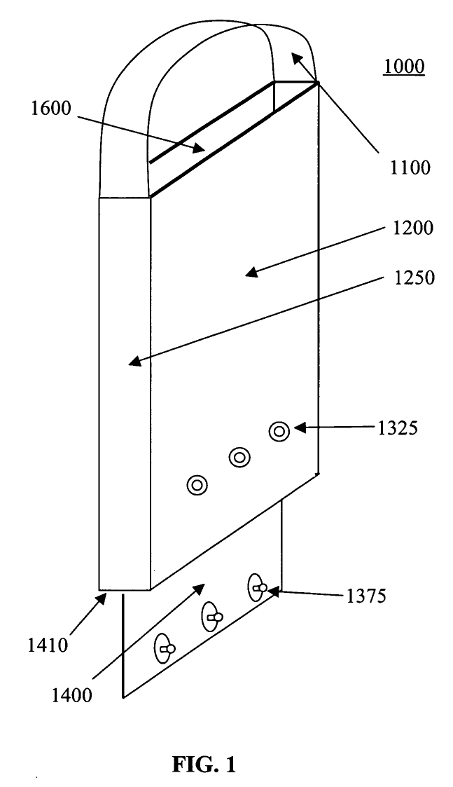

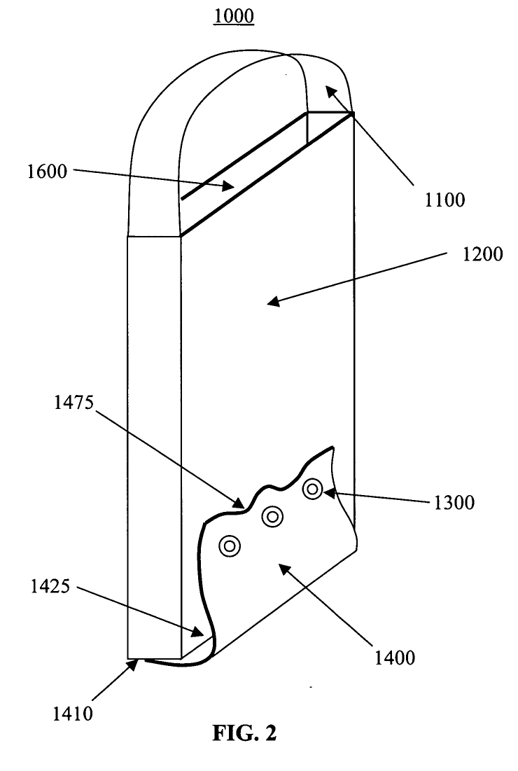

FIGS. 1 and 2 are perspective views of an exemplary embodiment of a container 1000, which can be used for storing and/or transporting medical devices and/or one or more other items. Container 1000 can, at least partially, enclose, conceal, store, protect, support, and/or transport, etc., a medical device, one or more other devices, and/or one or more items, etc.

Container 1000 can define a top opening 1600, which can be dimensioned to allow insertion and/or removal of a medical device and/or one or more other items, such as a snack, drink, purse, wallet, keys, instruction manual, magazine, book, cell phone, laptop computer, PDA, wiring, tube, flexible device, etc., through top opening 1600. A medical device can be a pump, a colostomy bag, a fluid bag, a catheter bag, a surgical food bag, a plasma bag, a blood product, a hydration source, an insulin source, a drug source, a monitor, a transmitter, a receiver, and/or a transceiver, etc.

FIG. 1 shows container 1000 in an open configuration, and FIG. 2 shows container 1000 in an closed configuration. Second portion 1400 can releasably couple to first portion 1200 at one or more locations offset from a bottom 1410 of container 1000. First portion 1200 and/or second portion 1400 can comprise a closure 1300. Closure 1300 can be a snap, zipper, button, pin, lacing, and/or hook- and/or loop- fastener, etc. Closure 1300 can comprise a first closure 1325 and/or a second closure 1375, which can releasably couple to form closure 1300.

First portion 1200 can releasably couple to second portion 1400 via closure 1300 to form sub-opening 1425 and/or sub-opening 1475. Second portion 1400, when releasably coupled to first portion 1200, and in a normal operative environment, can provide substantial support for a medical device and/or an other item. Sub-opening 1425 and/or sub-opening 1475 can be offset from bottom 1410 of container 1000. Sub-opening 1425 and/or sub-opening 1475 can be dimensioned such that a flexible device can pass through sub-opening 1425 and/or sub-opening 1475. Sub-opening 1425 and/or sub-opening 1475 can be dimensioned such that a medical device cannot pass through sub-opening 1425 and/or sub-opening 1475.

Container 1000 can comprise at least one strap 1100. Strap 1100 can be a shoulder strap, a hand strap, and/or a strap for hanging container 1000 on an IV-stand, etc. Strap 1100 can be releasably coupled and/or fixedly attached to first portion 1200 and/or second portion 1400. In a normal operating environment, strap 1100 can be pulled in a direction opposing bottom 1410 to facilitate carrying, transport, and/or hanging, etc., of the container 1000.

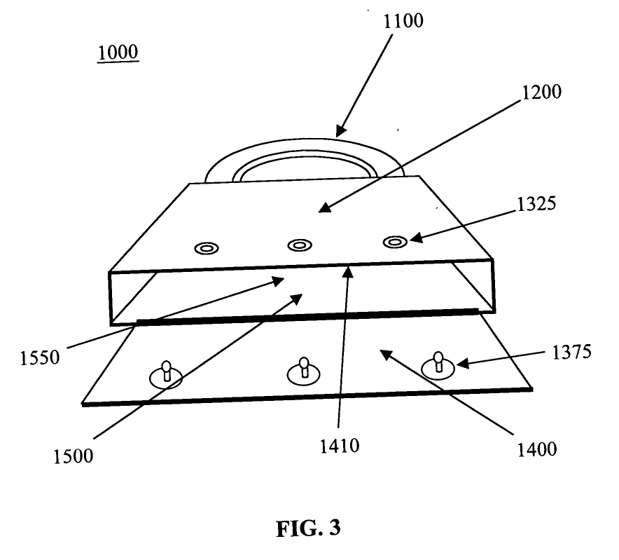

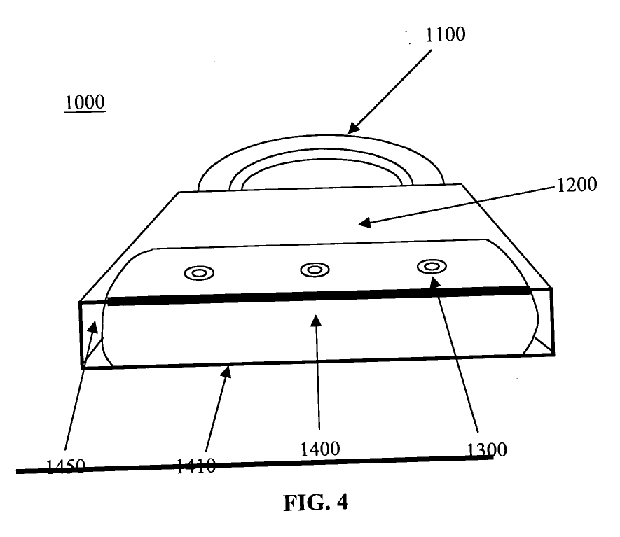

FIGS. 3 and 4 are perspective views of an exemplary embodiment of a container 1000. Container can define a bottom opening 1500 adjacent first portion 1200 and second portion 1400. Bottom opening 1500 can be dimensioned to allow a medical device and/or one or more other items to pass through bottom opening 1500.

Second portion 1400 can releasably couple to first portion 1200 to close bottom opening 1500 and form sub-opening 1450. Second portion 1400, when closed, can be medical-device-supporting. Closure 1325 can releasably couple with closure 1375 to form closure 1300 and/or releasably couple first portion 1200 to second portion 1400. Sub-opening 1450 can be offset from bottom 1410 of container. Sub-opening 1450 can be dimensioned to allow a flexible device to pass through sub-opening 1450. Sub-opening 1450 can be dimensioned such that a medical device cannot pass through sub-opening 1450. An area of bottom opening 1500 can be between approximately 1.0 square inches and approximately 80 square inches, including all values and subranges therebetween. An area of sub-opening 1450 can be between 0.01 and 40.0 square inches, including all values and subranges therebetween. A ratio of an area of sub-opening 1450 to an area of bottom opening 1500 can be between approximately 0.05 and approximately 0.35, including all values and subranges therebetween.

Interior 1550 can be adapted to at least partially, hold, protect, carry, house, enclose, store, conceal, and/or transport, etc., a medical device and/or one or more other items. Interior 1550 defines an interior volume that can be large enough to at least partially enclose a medical device and/or one or more other items. The interior volume can be between approximately 0.01 cubic feet and approximately 1.0 cubic feet, including all values and subranges therebetween.

FIG. 3 shows container 1000 in an open configuration, and FIG. 4 shows container 1000 in an closed configuration.

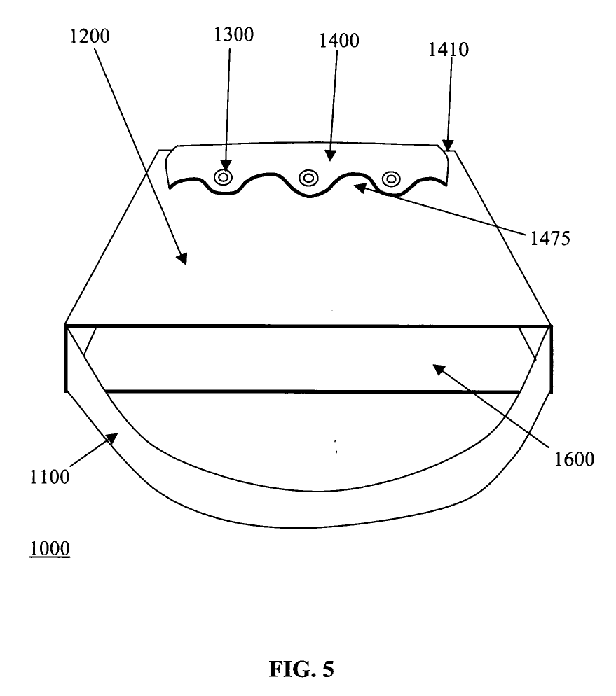

FIG. 5 is a perspective view of an exemplary embodiment of a container 1000. Second portion 1400, when releasably coupled to first portion 1200, and first portion 1200 can define a sub-opening 1475. Sub-opening 1475 can be offset from bottom 1410 of said container. Sub-opening 1475 can be dimensioned to allow a flexible device to pass through sub-opening 1475. Sub-opening 1475 can be dimensioned such that a medical device cannot pass through sub-opening 1475. A ratio of an area of sub-opening 1475 to an area of bottom opening 1500 (see FIG. 3) between approximately 0.05 and approximately 0.35, including all values and subranges therebetween.

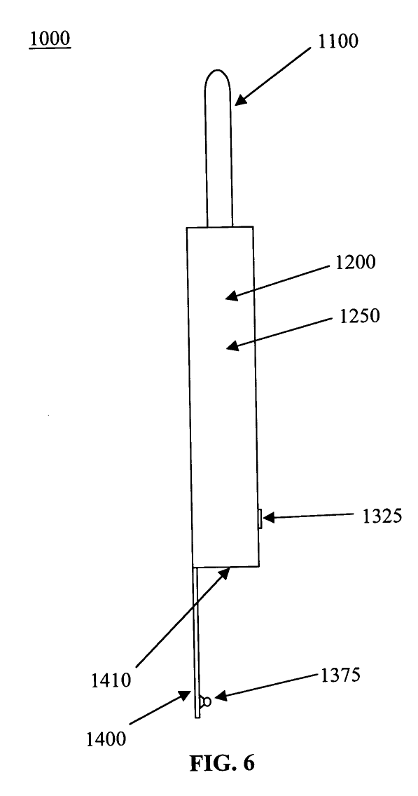

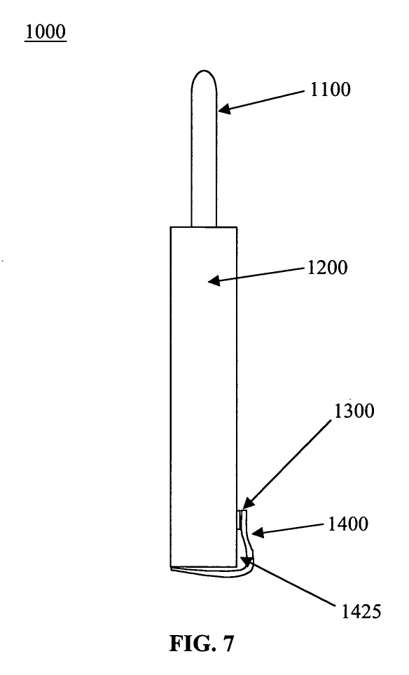

FIGS. 6 and 7 are side views of an exemplary embodiment of a container 1000. FIG. 6 shows container 1000 in a open configuration, and FIG. 7 shows container 1000 in a closed configuration.

As shown, first portion 1200 and/or second portion can comprise opposing lateral sides 1250. Closure 1325 can releasably couple to closure 1375 to form closure 1300. First portion 1200 can releasably couple to second portion 1400 via closure 1300 to form sub-opening 1425. Second portion 1400, when releasably coupled to first portion 1200, and in a normal operative environment, can provide substantial support for a medical device and/or one or more items. Sub-opening 1425 can be offset from bottom 1410 of container 1000. Sub-opening 1425 can be dimensioned such that a flexible device can pass through sub-opening 1425. Sub-opening 1425 can be dimensioned such that a medical device cannot pass through sub-opening 1425. A ratio of an area of sub-opening 1425 to an area of bottom opening 1500 (see FIG. 3) can be between approximately 0.05 and approximately 0.35, including all values and subranges therebetween.

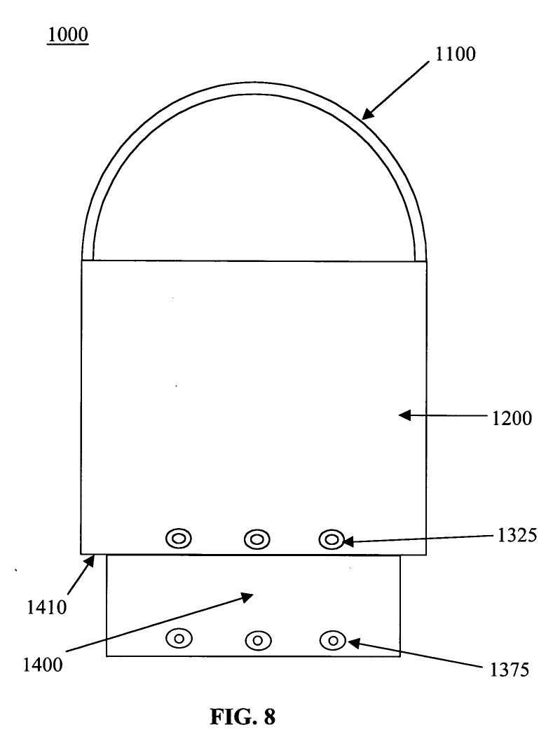

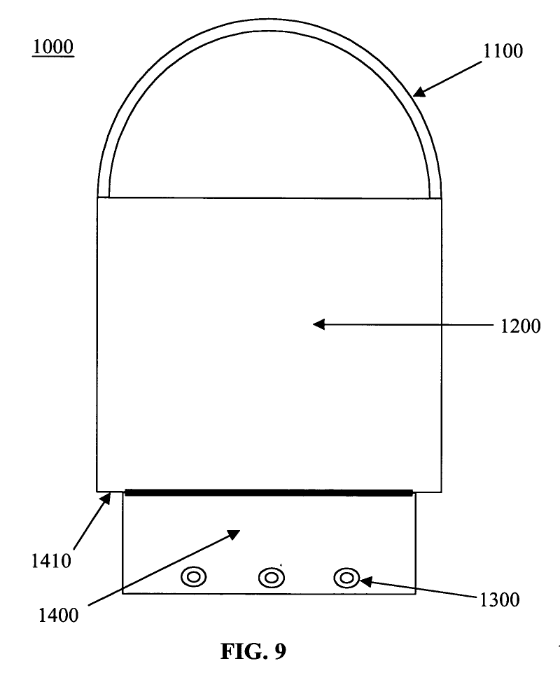

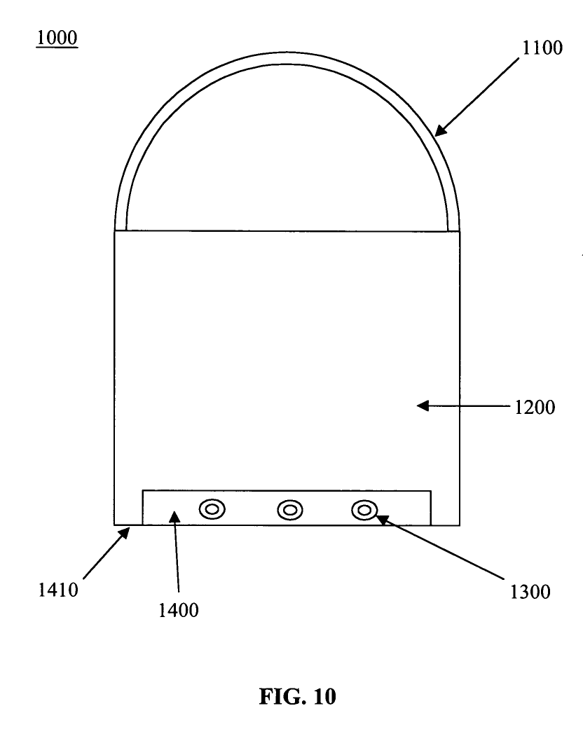

FIG. 8 is a front view, FIG. 9 is a front view, and FIG. 10 is a rear view of an exemplary embodiment of a container 1000. Container 1000 can be constructed of material, such as cotton, leather, vinyl, nylon, polyester, etc. The material can be unabrasive, soft, and/or relatively smooth. The material can feel relatively comfortable when it contacts the skin of a medical patient.

Container 1000 can be of a one-piece and/or multi-piece design. Container 1000 can comprise a first portion 1200 and/or a second portion 1400. Second portion 1400 can releasably couple to first portion 1200, such as on and/or at a front of container 1000. FIGS. 8 and 9 show container 1000 in an open configuration, and FIG. 10 shows container 1000 in a closed configuration.

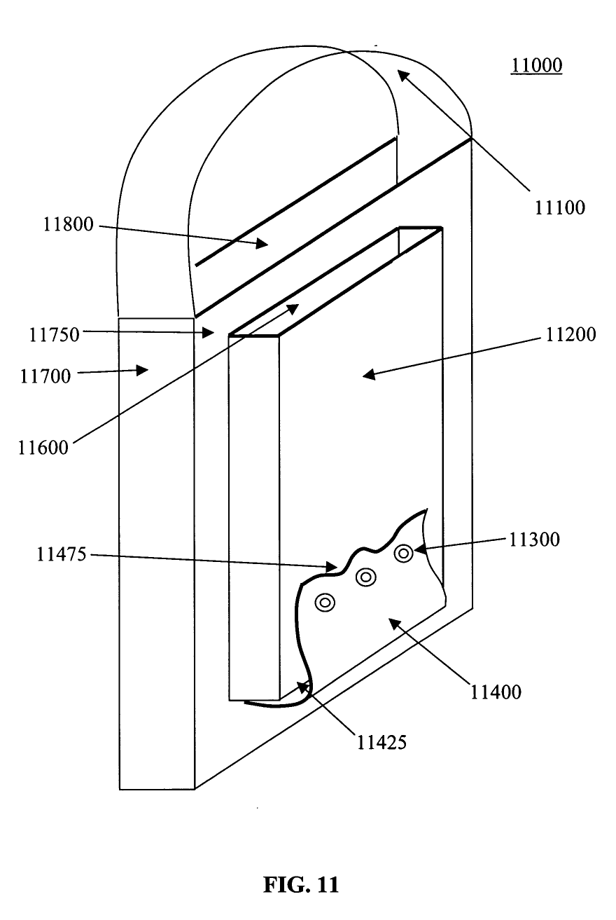

FIG. 11 is a perspective view of an exemplary embodiment of a container 11000. Container 11000 can be of single piece or multi-piece design. Container 11000 comprises a medical device pocket 11200 and an auxiliary device storage pocket 11700. Medical device pocket 11200 can be fixedly attached to an exterior wall 11750 of auxiliary storage pocket 11700. A ratio of an interior volume of medical device pocket 11200 and an interior volume of auxiliary storage pocket 11700 can be between approximately 0.25 and approximately 4.00, including all values therebetween, such as approximately 0.33, 0.51798, 0.99, 1.67, 3, 3.747, etc., and all subranges therebetween, such as from approximately 0.5 to approximately 2.499, from approximately 0.83 to approximately 1.83, etc. Medical device pocket 11200 and/or auxiliary storage pocket 11700 can comprise a releasably coupleable panel 11400. Releasably coupleable panel 11400 can be coupleable to medical device pocket 11200 at a location offset from a bottom 11410 of medical device pocket 11200. Releasably coupleable panel 11400, when releasably coupled to medical device pocket 11200, can form a sub-opening 11450. Medical device pocket 11200 and/or releasably coupleable panel 11400 can comprise a closure 11300. Closure 11300 can be releasably coupleable. Sub-opening 11450 can be dimensioned to allow a flexible device to pass through sub-opening 11450. Sub-opening 11450 can be dimensioned such that a medical device cannot pass through sub-opening 11450.

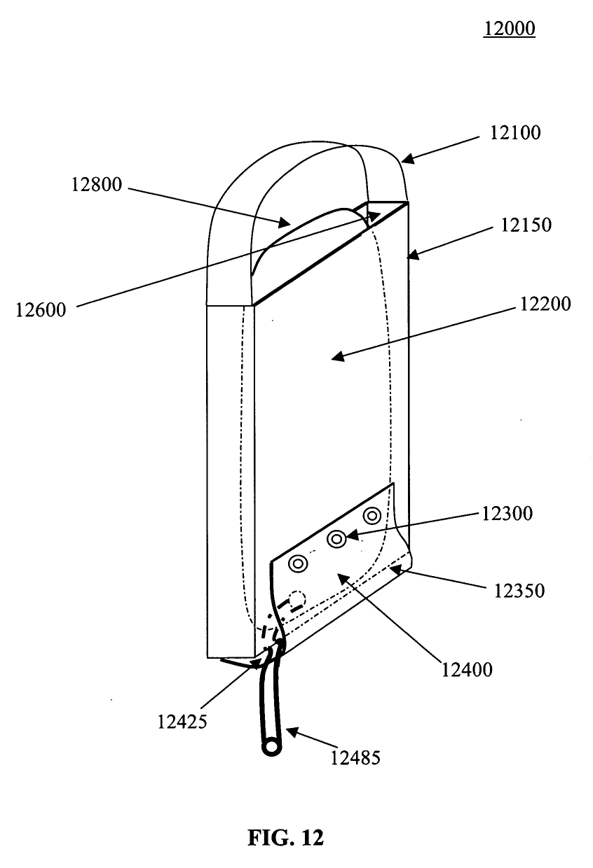

FIG. 12 shows an exemplary embodiment of a system 12000. System 12000 comprises a container 12050, a flexible device 12485, and/or a medical device 12800. Certain embodiments comprise a medical device 12800 coupled to a flexible device 12485 inserted through a bottom opening in container 12200. A second portion 12400 of a container can be releasably coupled to a first portion 12200 via a closure 12300. Second portion when releasably coupled, and/or first portion define a sub-opening 12450. Flexible device 12485 can pass through sub-opening 12450.

For example, if the flexible device is a feeding tube (see, e.g., element 12485 of FIG. 12) that is attached at one end to a medical feeding pump (see, e.g., element 12800 of FIG. 12), one can insert the feeding pump through a bottom opening in a container. One can then close a releasably coupleable panel of the container (see, e.g., element 12400 of FIG. 12), leaving the feeding tube protruding through a feeding tube hole in container (see, e.g. element 12450 of FIG. 12). The medical device and feeding tube can then be easily transported in the portable storage portable storage. A patient can carry the system by hand, over a shoulder, and/or hang it on an IV-stand, etc. The status of the feeding pump can also be monitored by viewing and/or removing it through a top opening in the container (see, e.g. element 12600 of FIG. 12) without decoupling the feeding tube at either end.

As another example, medical device 12800 can comprise a monitor, such as a glucose monitor, heart monitor, brain wave monitor, etc. The monitor can be coupled via a flexible device, such as a cable, wire, and/or optical fiber, to a sensor and/or actuator coupled to, attached on, and/or inserted into, a body of person. The monitor can be wirelessly coupled to a wireless information device that receives data from and/or provides data and/or instructions to, the monitor. The monitor can be coupleable via a wire, cable, and/or optical fiber to an information device.

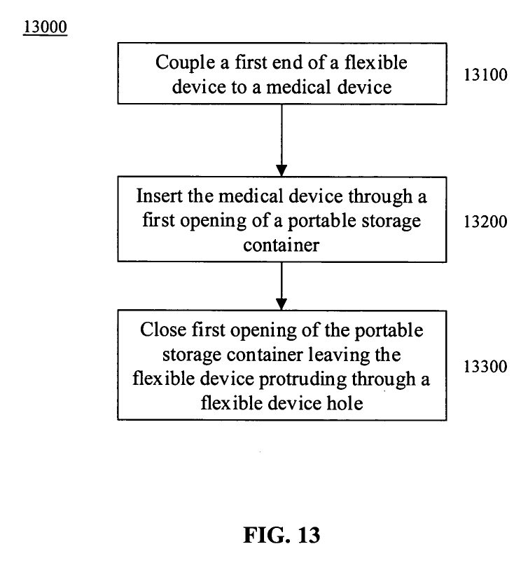

FIG. 13 is a flowchart of an exemplary method 13000 for using a container. At 13100, a first end of a flexible device can be coupled to a medical device. At activity 13200, the medical device coupled to the flexible device can be inserted through a first opening in a container. At activity 13300, the first opening of the container can be closed leaving the flexible device protruding from the container through a flexible device opening.

FIG. 13 represents only one of various methods of using a container. The container can be used to carry, hold, transport, and/or conceal, etc. various types of devices and/or objects. The container can be used with various types of flexible devices such as hoses, tubes, cords, wires, cables, wireless antennae, and/or ropes, etc. These flexible devices when used with a container are not limited to coupling with only medical devices.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all subranges therein. Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

1. A system, comprising:
a portable storage bag;
a medivice, herein defined as a medical device; and
a flexible medical tube coupled to said medivice;
said portable storage bag constructed of a soft, unabrasive, cloth material and adapted to transport said medivice,
said portable storage bag comprising an auxiliary storage pocket,
said portable storage bag further comprising a device pocket fixedly attached to an exterior wall of said auxiliary storage pocket,
said portable storage bag further comprising shoulder straps adapted to facilitate transport of said portable storage bag,
said auxiliary storage pocket comprising an open top, fixedly attached sides, and a fixedly attached underside,
said device pocket adapted to at least partially enclose said medivice, said device pocket comprising an open top, fixedly attached sides, and a releasably fastenable underside panel,
said device pocket defines a bottom opening,
said releasably fastenable underside panel adapted to close said bottom opening and releasably fasten to said device pocket offset from said bottom opening,
said releasably fastenable underside panel, when unfastened, allowing insertion of said medivice into, and removal of said medivice from, said device pocket,
said releasably fastenable underside panel, when fastened, defining a plurality of sub-openings, a first sub-opening from said plurality of sub-openings adapted to allow said flexible medical tube coupled to said medivice to pass through said first sub-opening,
said releasably fastenable underside panel, when fastened, defining a panel bottom,
at least one sub-opening from said plurality of sub-openings offset from said panel bottom,
said bottom opening, when open, substantially defining a bottom opening plane,
said bottom opening plane defining a first exit substantially perpendicular to said bottom opening plane,
said at least one sub-opening from said plurality of sub-openings defining a second exit substantially perpendicular to a sub-opening plane defined by said at least one sub-opening, said second exit oriented substantially opposite said first exit,
said first sub-opening dimensioned to prevent passage of said medivice through said first sub-opening,
said open top of said device pocket dimensioned to allow said medivice to pass through said open top of said device pocket,
said device pocket defining a device storage volume,
said auxiliary pocket defining an auxiliary storage volume,
a ratio of said device storage volume to said auxiliary storage volume in a range of approximately 0.25 to approximately 0.75,
said first sub-opening defining a tube opening area,
said bottom opening defining a device opening area,
a ratio of said tube opening area to said device opening area in a range of approximately 0.05 to approximately 0.35.

2. An apparatus, comprising:
a non-garment container for a medical device comprising:
a medical device storage pocket,
an auxiliary storage pocket,
at least one carrying strap coupled to said container,
a panel releasably couplable to said medical device storage pocket,
and a releasable closure attached to said medical device pocket and to said panel for closing a releasably closable first opening adjacent said medical device pocket and said panel, said medical device storage pocket coupled to said auxiliary storage pocket,
said medical device storage pocket defines a second opening opposing said releasably closable first opening, said releasably closable first opening, when open, dimensioned to allow the medical device to pass through said first opening,
said releasably closable first opening, when closed, dimensioned to prevent the medical device from passing through said first opening,
said releasably closable first opening closable to form a plurality of sub-openings,
said panel, when releasably coupled to said medical device storage pocket, defines a bottom,
said bottom, when said panel is open, substantially defining a bottom opening plane,
said bottom opening plane defining a first exit substantially perpendicular to said bottom opening plane,
at least one sub-opening from said plurality of sub-openings defining a second exit substantially perpendicular to a sub-opening plane defined by said at least one sub-opening, said second exit oriented substantially opposite said first exit,
at least one from said plurality of sub-openings offset from said bottom,
a first sub-opening from said plurality of sub-openings dimensioned to allow a flexible device to pass through said first sub-opening.

3. An apparatus, comprising:
a non-garment container for a medical device, said container comprising:
a carrying strap coupled to said container;
a medical-device-hiding portion;
a medical-device-supporting portion couplable to said medical-device-hiding portion;
an auxiliary storage portion; and
a releasable closure attached to said medical-device-hiding portion and to a second portion for closing a releasably closable first opening adjacent said medical-device-hiding portion and said medical-device-supporting portion;
said medical-device-hiding portion and said second portion defining a second opening opposing said first opening;
said releasably closable first opening, when open, dimensioned to allow the medical device to pass through said first opening;
said releasably closable first opening, when closed, dimensioned to prevent the medical device from passing through said first opening;
said second opening dimensioned to allow the medical device to pass through said second opening;
said first opening closable to form a plurality of sub-openings;
said second portion, when coupled to a first portion closing said first opening, defines a bottom;
said bottom, when releasably closable first opening is open, substantially defining a bottom opening plane,
said bottom opening plane defining a first exit substantially perpendicular to said bottom opening plane,
at least one sub-opening from said plurality of sub-openings defining a second exit substantially perpendicular to a sub-opening plane defined by said at least one sub-opening, said second exit oriented substantially opposite said first exit,
at least one of said plurality of sub-openings offset from said bottom;
a first sub-opening from said plurality of sub-openings dimensioned to allow a flexible device to pass through said first sub-opening.

4. The apparatus of claim 3, wherein said first sub-opening dimensioned to prevent passage of the medical device through said first sub-opening.

5. The apparatus of claim 3, wherein said container comprises a cloth material.

6. The apparatus of claim 3, wherein at least a portion of said container is insulated.

7. The apparatus of claim 3, said auxiliary storage portion defining an auxiliary opening, said auxiliary opening opposing said first opening.

8. The apparatus of claim 3, wherein said second opening is releasably closable.

9. The apparatus of claim 3, wherein said second opening is releasably closable, said second opening closable to form a plurality of top sub-openings, a first top sub-opening of said top sub-openings dimensioned to allow at least one flexible device to pass through said first top sub-opening, said first top sub-opening dimensioned to prevent passage of the medical device.

10. The apparatus of claim 3, wherein said first portion and said second portion, when closed, define an interior volume of less than approximately 1.0 cubic feet.

11. The apparatus of claim 3, wherein said first portion and said second portion, when closed, define an interior volume of greater than approximately 0.01 cubic feet.

12. The apparatus of claim 3, said first portion and said second portion, when closed, defining a first interior volume, said auxiliary storage portion defining a second interior volume, a ratio of said first interior volume to said second interior volume between approximately 0.25 and approximately 4.0.

13. The apparatus of claim 3, wherein an area defined by said first opening is between approximately 1.0 square inch and approximately 80.0 square inches.

14. The apparatus of claim 3, wherein an area of said first sub-opening is between approximately 0.01 square inches and approximately 40.0 square inches.

15. The apparatus of claim 3, wherein a ratio of an area said first sub-opening to said first opening is between approximately 0.05 and approximately 0.35.

16. The apparatus of claim 3, wherein said second portion releasably couples to said first portion at a location offset from said bottom of said second portion.

17. A method, comprising a plurality of activities comprising:
inserting a medical device into a container, said container comprising a medical device pocket, said container comprising an auxiliary storage pocket, said medical device pocket coupled to said auxiliary storage pocket, said medical device pocket adapted to transport at least a portion of said medical device, said medical device pocket adapted to at least partially enclose said medical device, said container comprising a carrying strap, said medical device pocket defining a releasably closable first opening and an opposing second opening, said first opening defining a bottom opening plane, said first opening closes to form a plurality of sub-openings, said bottom opening plane defining a first exit substantially perpendicular to said bottom opening plane, at least one sub-opening from said plurality of sub-openings defining a second exit substantially perpendicular to a sub-opening plane defined by said at least one sub-opening, said second exit oriented substantially opposite said first exit, a first sub-opening from said plurality of sub-openings dimensioned to allow a flexible device to pass through said first sub-opening, and
coupling said flexible device to said medical device.

18. The method of claim 17, further comprising closing said first opening.

19. The method of claim 17, further comprising closing said first opening and extending said flexible device through said first sub-opening.

20. The method of claim 17, further comprising closing said first opening around said flexible device, said flexible device extending through said first sub-opening.

21. A system comprising:
a medical device;
a flexible device; and
a container;
said container comprising a carrying strap,
said container comprising an auxiliary storage pocket,
said container comprising a medical device storage pocket coupled to said auxiliary storage pocket,
said container adapted to at least partially enclose said medical device;
said container defining a releasably closable first opening and an opposing second opening,
said first opening, when closed, forming a plurality of sub-openings,
said first opening, when closed defining a bottom,
said bottom, when said first opening is open, substantially defining a bottom opening plane,
said bottom opening plane defining a first exit substantially perpendicular to said bottom opening plane,
at least one sub-opening from said plurality of sub-openings defining a second exit substantially perpendicular to a sub-opening plane defined by said at least one sub-opening, said second exit oriented substantially opposite said first exit,
a first sub-opening from said plurality of sub-openings dimensioned to allow said flexible device to pass through said first sub-opening.
